(19) United States
(12) Patent Application Publication
(10) Pub. No.: US 2022/0395640 A1
(43) Pub. Date: Dec. 15, 2022

(54) MEDICAMENT DELIVERY DEVICE AND MEDICAMENT DELIVERY DEVICE ADD-ON

(71) Applicant: QUIO TECHNOLOGIES LLC, NEW YORK, NY (US)

(72) Inventor: Jared Schwartzentruber, Astoria, NY (US)

(21) Appl. No.: 17/776,320

(22) PCT Filed: Nov. 3, 2020
(86) PCT No.: PCT/IB2020/060308

§ 371 Date: May 12, 2022

Related U.S. Application Data
(60) Provisional application No. 62/933,996, filed on Nov. 12, 2019.

(30) Foreign Application Priority Data
Dec. 20, 2019 (EP) 19218579.1

(51) Int. Cl. A61M 5/20; A61M 5/315; A61M 5/32

(52) U.S. Cl. CPC A61M 5/20; A61M 5/31566; A61M 5/3202; A61M 2005/2006; A61M 2205/3368; A61M 2205/52

(57) ABSTRACT

A device is presented having an electronic circuit, where the device can be a medicament delivery device or a medicament delivery device add-on component that includes an electronic circuit having a temperature sensor configured to measure a temperature, where the electronic circuit is configured to passively remain in a first mode when the temperature is on a first side of a set temperature and to transition to a second mode when the temperature transitions to a second side of the set temperature. In some embodiments, the electronic circuit can have one or more of a feedback portion configured to switch from a first state to a second state in response to the electronic circuit transitioning from the first mode to the second mode, a lock, and a recordal portion to record when the electronic circuit transitions from the first mode to the second mode.

### CROSS REFERENCE TO RELATED APPLICATIONS

The present application is a U.S. National Phase Application pursuant to 35 U.S.C. § 371 of International Application No. PCT/IB2020/060308 filed Nov. 3, 2020, which claims priority to U.S. Provisional Patent Application No. 62/933,996 filed Nov. 12, 2019, and European Patent Application 19218579.1, filed Dec. 20, 2019. The entire disclosure contents of these applications are herewith incorporated by reference into the present application.

### TECHNICAL FIELD

The present disclosure concerns medicament delivery devices and medicament delivery device add-ons, and particularly medicament delivery devices and medicament delivery device add-ons comprising a temperature sensor.

### BACKGROUND

There is currently a trend in many industries towards smart and connected devices, for example to improve user experience or to improve tracking of devices through a supply chain. Smart and/or connected devices are also of interest in the medical device industry, but in medicament delivery devices such as auto-injectors and pen injectors, developing smart and/or connected devices that meet practical requirements with regards to parameters such as size, cost, battery life and recyclability is proving challenging. The applicant has appreciated that improvements can be made in comparison to existing devices.

### SUMMARY

The present disclosure is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

A first aspect concerns a device comprising an electronic circuit, wherein the device is a medicament delivery device or a medicament delivery device add-on, the electronic circuit comprising a temperature sensor configured to measure a temperature of the device; wherein the electronic circuit is configured to passively remain in a first mode when the temperature is on a first side of a set temperature and to transition to a second mode when the temperature transitions to a second side of the set temperature. In one embodiment, the electronic circuit comprises a feedback portion configured to switch from a first state to a second state in response to the electronic circuit transitioning from the first mode to the second mode, so as to provide information related to the temperature to a user. In one embodiment, the electronic circuit comprises a lock configured to switch from a first state to a second state in response to the electronic circuit transitioning from the first mode to the second mode. In one embodiment, the electronic circuit comprises a recordal portion to record when the electronic circuit transitions from the first mode to the second mode.

In developing embedded or add-on connectivity for injectable therapies, it is beneficial that the electronics remain off when not in use. This can help the electronics to remain useable during the full lifecycle of the drug product, during which a medicament delivery device could sit on a shelf for years before being used.

The first mode is typically a passive mode, in which little or no current flows through the circuit, and the second mode is typically an active mode, in which current flows through the circuit. The electronic circuit is configured to passively remain in a first mode. This means that the device does not need to periodically measure the temperature, and that a user does not need to interact with the device (for example with a button or a switch on the device) to activate the electronic circuit, as the electronic circuit can activate itself automatically. Being passive can be beneficial because it can minimise power use (although the power draw in the quiescent state would typically be slightly above zero due to current leakage) and can therefore minimise the size and/or cost of a corresponding power source. It can also avoid the need for a user to activate the electronic circuit from a first (passive) mode to a second (active) mode, which can simplify use of the device for the user. Being able to check the device between manufacture and use, such as during transport or at the pharmacy before being dispensed to a patient, can also be useful in terms of checking whether a drug has already been kept at an inappropriate temperature during transport or storage.

For some medicaments, for example some injectable biologics, the drug should be kept at refrigeration temperatures at all stages between manufacture and use to ensure the stability of the drug product. When the patient is ready to administer their injectable dose, they are typically instructed to remove the injectable from the refrigerator and wait 30 minutes for the device to warm prior to injection. When the drug product is cold, the viscosity is increased, which may increase the pain of injection. This wait time is intended to reduce this pain. Providing an indication of when the temperature is appropriate for injection can therefore improve user experience.

The user can be an end user, such as a patient who is injecting themselves or a caregiver injecting a patient. In some embodiments, someone could check the state of the device before it reaches an end user, for example someone could check the device during transport or in a pharmacy before dispensing the device. In such cases, any person or machine who can check the feedback portion would also be a user in the sense of using the feedback portion to receive temperature-related information.

Providing a device that can indicate when the temperature of the device has gone above or below an acceptable level can avoid use of drugs with a reduced efficacy due to temperature-related degradation during storage or transport. This can improve patient outcomes.

In one embodiment, the electronic circuit is configured to passively remain in a first mode when the temperature is below a set temperature and to transition to a second mode when the temperature transitions to above the set temperature. In one embodiment, the electronic circuit is configured to passively remain in a first mode when the temperature is above a set temperature and to transition to a second mode when the temperature transitions to below the set temperature.

In one embodiment, the electronic circuit comprises a feedback portion. In one embodiment, the feedback portion is configured to switch to the second state when the temperature is on the second side of the set temperature and is configured to return to the first state if the temperature subsequently returns to a temperature on the first side of the set temperature. In one embodiment, the feedback portion is configured to switch to the second state when the temperature is on the second side of the set temperature and is configured to remain in the second state if the temperature subsequently returns to a temperature on the first side of the set temperature. Providing a feedback portion may help a user with correct use of the device. Providing a feedback portion may additionally or alternatively help indicate whether a device has been kept at an appropriate temperature in the past.

In one embodiment, the electronic circuit comprises a lock. In one embodiment, the lock is configured to switch to the second state when the temperature is on the second side of the set temperature and is configured to return to the first state if the temperature subsequently returns to a temperature on the first side of the set temperature. In one embodiment, the lock is configured to switch to the second state when the temperature is on the second side of the set temperature and is configured to remain in the second state if the temperature subsequently returns to a temperature on the first side of the set temperature. In one embodiment, the lock is unlocked in the first state and locked in the second state. In one embodiment, the lock is unlocked in the second state and locked in the first state. Providing a lock can improve patient outcomes, as it can make incorrect use of the device harder, for example by stopping a user from using a drug while the temperature is below a minimum injection temperature, and/or stopping a user from using a drug that may no longer be effective due to a past temperature excursion beyond an allowed temperature limit or beyond an allowed temperature limit for more than an allowed time.

In one embodiment, the set temperature is a temperature between −10 and 100 degrees Celsius. In one embodiment, the set temperature is a temperature between −10 and 10 degrees Celsius, between 5 and 25 degrees Celsius, or between 25 and 50 degrees Celsius. In one embodiment, the temperature sensor is configured to measure a temperature reflective of an ambient temperature, of a temperature of a medicament within the device. In one embodiment, the temperature sensor is configured to measure a temperature of the device. In one embodiment, the temperature sensor is configured to measure a temperature of a user.

In one embodiment, the temperature sensor is on an outer portion of the device such that the temperature sensor. In one embodiment, the temperature sensor is configured to measure the temperature of a user. In one embodiment, the device comprises a medicament delivery device, and wherein the outer portion of the device is part of a housing, an outer shell, a button, a grip or a medicament delivery member guard of the medicament delivery device. In one embodiment, the device comprises a medicament delivery device add-on, and the outer portion of the device is part of an outer housing, a button or a grip of the medicament delivery device add-on. In one embodiment, the outer portion of the device is a medicament delivery member guard of the medicament delivery device and the medicament delivery member guard extends from a proximal end to a distal end, wherein the proximal end is the end closest to the injection site when in use, and wherein the temperature sensor is at the proximal end of the medicament delivery member guard. In one embodiment, the set temperature is a temperature between 20 and 37 degrees Celsius, or between 25 and 35 degrees Celsius, or between 30 and 33 degrees Celsius. These embodiments can provide a temperature sensor on the outside of the device to measure the temperature of the user, allowing the presence of a user to trigger a change in a lock or a feedback portion.

In one embodiment, the temperature sensor is a thermistor. In one embodiment, the electronic circuit comprises a voltage comparator. In one embodiment, the voltage comparator comprises one or more of a transistor or an operational amplifier. In one embodiment, the electronic circuit comprises an integrated circuit temperature sensor, such as the LM26LV temperature switch from Texas Instruments. These embodiments can provide a simple circuit design with off-the-shelf electronics to minimise cost.

In one embodiment, the medicament delivery device is an auto-injector. In one embodiment, the medicament delivery device add-on is an auto-injector add-on.

In one embodiment, the feedback portion provides feedback in one or more of a tactile manner, an audible manner and a visual manner. In one embodiment, the feedback portion comprises one or more of a display, a buzzer, a light and a switch.

In one embodiment, the set temperature is a temperature above which a drug may be injected. In one embodiment, the set temperature is a temperature above which or a temperature below which a drug is no longer useable.

A second aspect concerns an electronic unit of a medicament delivery device or a medicament delivery device add-on, the electronic unit being configured to passively remain in a first mode when the temperature is on a first side of a set temperature and to transition to a second mode when the temperature transitions to a second side of the set temperature. In one embodiment, the electronic unit comprises a feedback portion configured to switch from a first state to a second state in response to the electronic unit transitioning from the first mode to the second mode, so as to provide information related to the temperature to a user; and/or a lock configured to switch from a first state to a second state in response to the electronic unit transitioning from the first mode to the second mode; and/or a recordal portion to record when the electronic circuit transitions from the first mode to the second mode.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the accompanying drawings, in which:

FIG. 1 shows a perspective view of a medicament delivery device;

FIG. 2 shows a circuit diagram of an electronic circuit;

FIG. 3 shows a circuit diagram of a second electronic circuit;

FIG. 4 shows a cap with feedback portion when an electronic circuit is in an inactive mode;

FIG. 5 shows a cap with feedback portion when an electronic circuit is in an active mode;

FIG. 6 shows an alternative cap with feedback portion when an electronic circuit is in an inactive mode; and

FIG. 7 shows an alternative cap with feedback portion when an electronic circuit is in an active mode.

### DETAILED DESCRIPTION

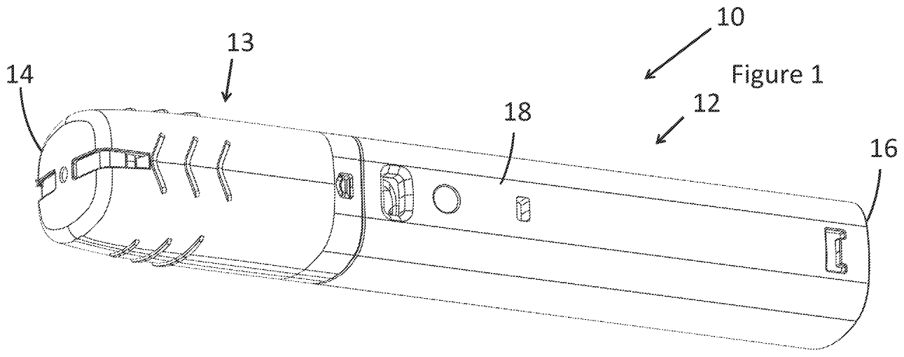

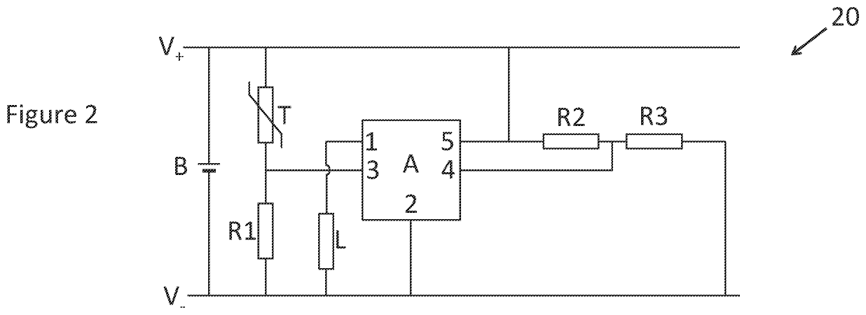

A medicament delivery device 10 is shown in FIG. 1. The medicament delivery device comprises an electronic circuit (not shown in FIG. 1; example electronic circuits are shown in FIGS. 2 and 3 and described below). The electronic circuit comprises a temperature sensor configured to measure a temperature. The electronic circuit is configured to passively remain in a first mode when the temperature is on a first side of a set temperature and to transition to a second mode when the temperature transitions to a second side of the set temperature. The electronic circuit can comprise various features, for example a feedback portion configured to switch from a first state to a second state in response to the electronic circuit transitioning from the first mode to the second mode, so as to provide information related to the temperature to a user; and/or the electronic circuit comprises a lock configured to switch from a first state to a second state in response to the electronic circuit transitioning from the first mode to the second mode, and/or a recordal portion to record when the electronic circuit transitions from the first mode to the second mode.

One example of a medicament delivery device is shown in FIG. 1, but the systems described herein may be used with various types and shapes of medicament delivery devices or medicament delivery device add-ons. In the case of a medicament delivery device, the medicament delivery device may be an auto-injector or a pen injector, for example. In the case of a medicament delivery device add-on, the medicament delivery device add-on may be for an auto-injector or a pen injector, for example. FIG. 1 shows a medicament delivery device 10 that could comprise an electronic circuit as disclosed herein. The medicament delivery device 10 comprises a body 12 and a cap 13. The medicament delivery device 10 extends from a proximal end 14 (first end) to a distal end 16 (second end). The body 12 comprises an outer housing 18. Although not all components are shown in FIG. 1, the medicament delivery device would typically also include components such as a medicament container inside the outer housing 18 of the body 12 and a powerpack assembly inside the outer housing 18 of the body 12. The medicament container typically comprises a medicament barrel (inside which a medicament can be placed) and a needle with a needle cover. The cap 13 typically comprises a needle cover remover. The medicament delivery device add-on is typically configured to attach to the cap and/or the body of a medicament delivery device. In some embodiments, the medicament delivery device comprises a medicament delivery device add-on; in such embodiments, part of the electronic circuit may be in a medicament delivery device and part of the electronic circuit may be in the medicament delivery device add-on of the medicament delivery device.

In the example electronic circuit 20 in FIG. 2, a battery B is provided to create a voltage difference between a low voltage V₋ and a high voltage V₊. A voltage comparator A is connected across the voltage difference between the voltage V₋ and the voltage V₊. The voltage comparator has five ports A1, A2, A3, A4, A5. The ports can alternatively be denoted as A1=OUT, A2=GND (ground), A3=IN+, A4=IN−, and A5=VCC (common collector voltage). A load L is connected between V₋ and A1. A5 is connected directly to V₊. A first voltage divider comprises the temperature sensor T and a first resistor R1, with T connected between V₊ and R1, and R1 connected between T and V₋. A3 is directly connected to the first voltage divider between T and R1. Similarly, a second voltage divider comprises a second resistor R2 and a third resistor R3. R2 is directly connected between V₊ and R3, and R3 is directly connected between R2 and V. A4 is directly connected to the second voltage divider between R2 and R3.

FIG. 3 shows an alternative electronic circuit 21. Instead of a voltage comparator, a transistor TR is provided. As with the example in FIG. 2, battery B is provided to create a voltage difference between a low voltage V₋ and a high voltage V₊, and a voltage divider comprises the temperature sensor T and a fourth resistor R4, with T connected between V₊ and R4, and R4 connected between T and V₋. The base of the transistor is directly connected to the voltage divider between R4 and T. A fifth resistor R5 is connected between V₊ and the collector of the transistor, and a load L is connected between the emitter of the transistor and V₋.

FIGS. 2 and 3 show examples of electronic circuits, and such electronic circuits could be altered in various ways within the scope of the present disclosure, with different components and/or different combinations of resistors. For example, the transistor TR shown in FIG. 3 is an npn transistor, although a pnp transistor could also be used. The transistor may be a MOSFET. Rather than an operational amplifier or a transistor as shown in FIG. 2 and FIG. 3 respectively, other components or circuits that result in a similar or identical result could be used, such as other types of voltage comparator, voltage amplifier or other combinations of resistors to provide alternative forms of voltage dividers. Further components could also be included in the electronic circuit, such as a recordal portion to record data and/or a connectivity portion to transmit data to another device, for example to a mobile phone or a computer using Bluetooth or Wifi, or to transmit data to the cloud, for example to a cloud database, using a wired or wireless network such as a broadband network or a mobile phone network. Resistor values for resistors such as R1 to R5 can be varied depending on circuit design, but would typically be set to keep quiescent current draw as low as possible while the electronic circuit is inactive, to minimise quiescent loss of power while the device is in transport or storage.

The temperature sensor T may be a thermistor or any other suitable temperature sensor with a resistance that varies depending on temperature. As a result, the electronic circuit can be arranged to draw almost no current below the set temperature, and then to draw significantly more current above the set temperature, since the resistance of the temperature sensor has changed. With more current passed through the load (see for example load L in FIGS. 2 and 3), components in the load such as components related to device connectivity, additional sensors, screens and buzzers can function. Additional sensors could include an IMU sensor (inertial measurement unit sensor), for example.

The temperature measured by a temperature sensor in the devices described herein is a temperature of the device. The temperature measured by such a temperature sensor is typically the temperature of the temperature sensor itself. Depending on the device and the location of the temperature sensor within the device, the temperature may closely reflect (e.g. within 1 degree Celsius) the temperature of, for example, part of a medicament delivery device add-on, a medicament delivery device cap, a medicament delivery device outer shell, a medicament container, a medicament barrel or a medicament itself. Different parts of a medicament delivery device or medicament delivery device add-on may have different temperatures at different times; for example, if a medicament delivery device has been at a constant temperature of 5 degrees Celsius in a fridge for a period of 24 hours, all the parts of the medicament delivery device would normally be at 5 degrees Celsius. If the medicament delivery device is then removed from the fridge and placed in an environment at 20 degrees Celsius, different parts may warm up at different rates. For example, 10 minutes after removal from the fridge, the outer shell may be at 15 degrees Celsius and the medicament itself at 10 degrees Celsius. In this example, if it is desired that the electronic circuit transitions from a first mode to a second mode when the medicament temperature is about 10 degrees Celsius, but the temperature sensor is attached to the outer shell and therefore closely reflects the temperature of the outer shell, then the set temperature could be set at 15 degrees Celsius. In other words, the set temperature may need to be set at a temperature offset from the actual medicament temperature at the point that the electronic circuit is intended to transition. In general, therefore, the set temperature at which the electronic circuit is designed to transition from a first mode to a second mode may differ from the actual temperature of the medicament at the point when the electronic circuit is designed to transition, depending on how closely the temperature of the temperature sensor reflects the temperature of the medicament. This can help account for expected differences in temperature between different parts of a device. The temperature of the temperature sensor could additionally or alternatively reflect an ambient temperature of the environment around the device or the temperature of a user, for example when the temperature sensor is on an outer portion of the device.

A low-dropout regulatory (LDO) may also be included, particularly if a battery with an unstable discharge voltage is used. Although a battery B is shown in FIGS. 2 and 3, other types of power storage or power source may also be used, such as capacitors or mains electricity via a cable. In cases where a finite power source such as a battery is provided without any provision of a means for recharging the device, the power supply can eventually run out if the electronic circuit is on for too long, which can additionally provide an indication of whether the temperature has been outside of tolerated levels for too long. For example, if the set temperature is 10 degrees Celsius and the electronic circuit is configured to transition from the first mode to the second mode when the temperature goes from below 10 degrees Celsius to above 10 degrees Celsius, and the electronic circuit is drawing power for a month because the temperature of the device has been at 20 degrees Celsius for a month, the power source may run out. The user will then no longer see any change in the feedback portion, even if they provide a temperature change from 5 degrees Celsius to 20 degrees Celsius with the intention of using the device. In such a case, the user could be instructed to dispose of the device without using the device.

Load L can include, for example, a feedback portion that provides feedback to a user or a lock. The feedback portion can provide audible, visible and/or tactile feedback, for example, so that a user receives an indication that the device is ready to use once the device reaches a set temperature. As such, the feedback portion may provide an indication of the current temperature of the device, such as by displaying a temperature reading, by displaying a message, or changing in some other way. Alternatively, the indication to the user may provide an indication of the previous temperature of the device. For example, a display window may be flipped from showing a green indicator to showing a red indicator if the temperature has increased beyond a set temperature between manufacture of the device and the time that the user looks at the device immediately before use. Further details on possible feedback portions are outlined below.

The set temperature can be fixed at the time of manufacture, or could also be adjustable if variable resistors are provided instead of fixed-value resistors, for example. The value chosen for the set temperature can vary depending on the desired effect; for example, the set temperature could be between −50 and 100 degrees Celsius. For example, a drug delivery set temperature could be chosen for an electronic circuit designed to provide information on whether the drug has reached an appropriate temperature for injection. For example, the set temperature could be between 0 and 30 degrees Celsius, between 5 and 25 degrees Celsius, between 10 and 20 degrees Celsius or between 8 and 12 degrees Celsius. The set temperature could alternatively be fixed at a temperature above which a medicament delivery device should no longer be used, for example because the efficacy of a drug in the medicament delivery device is lost or degraded when the drug has been heated above a certain temperature. For example, the set temperature could then be between 5 and 100 degrees Celsius, between 15 and 75 degrees Celsius, between 25 and 50 degrees Celsius or between 30 and 40 degrees Celsius. The set temperature could alternatively be fixed at a temperature below which a medicament delivery device should no longer be used, for example because the efficacy of a drug in the medicament delivery device is lost or degraded when the drug has been cooled below a certain temperature. For example, the set temperature could then be between −50 and 25 degrees Celsius, or between −20 and 20 degrees Celsius, or between −10 and 5 degrees Celsius. In some devices, there is a risk of the injection stalling in low temperature conditions, for example because the medicament is too viscous for the device when the medicament is below a certain temperature. The set temperature could therefore also be set at a temperature above which there is little or no risk of the device stalling.

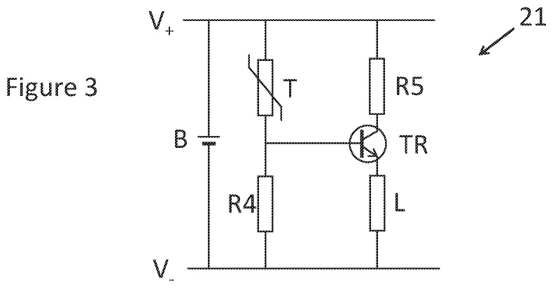

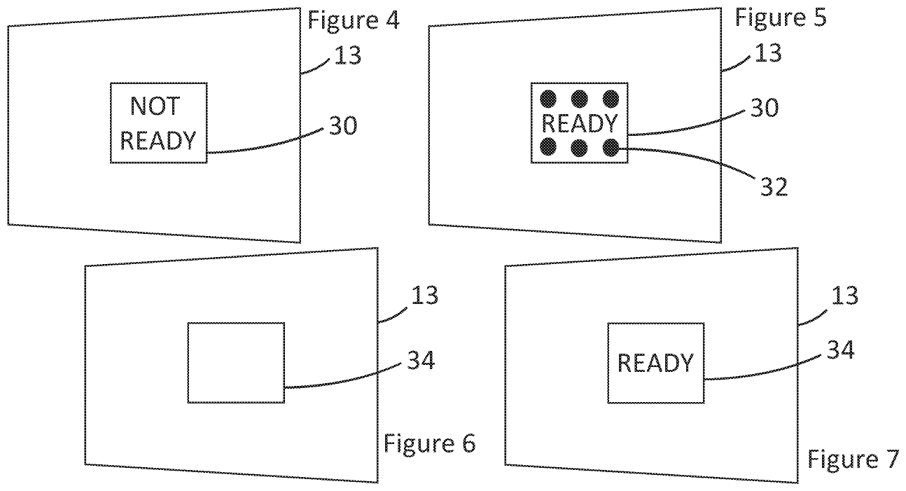

FIGS. 4 and 5 show an example of a feedback portion 30. In an inactive mode of the electronic circuit, the feedback portion remains in a first state in which the feedback portion displays a panel showing the words ‘NOT READY’. Once the temperature has risen above the set temperature, the panel showing the words ‘NOT READY’ is moved out of the view, and another panel showing the word ‘READY’ and protrusions 32 is moved into view, thus providing the feedback portion in a second state. The feedback portion in the second state gives both tactile and visual indications that the device is now ready for use, although in alternative embodiments just the tactile indication or just the visual indication could be provided. Although not shown in FIGS. 4 and 5, opposing ribs on the feedback portion 30 and the cap 13 could also be provided that impact one another as the ‘NOT READY’ panel moves away and the ‘READY’ panel moves into view, additionally providing an audible indication.

FIGS. 6 and 7 show an alternative indicator. In this embodiment, a display screen 34 is provided. When the electronic circuit is in an inactive mode, the display is blank. Once the electronic circuit switches to an active mode, the display shows the word ‘READY’.

Numerous types of feedback portion may be provided, either individually or in combination, depending for example on cost limitations, the size and shape of the medicament delivery device and on the needs of the user group. A feedback portion would typically provide one or more of a visual, a tactile and an audible indication, although any form of feedback that can be sensed by a user could be used. In FIGS. 4 and 5, an example of an analogue screen is shown (that is, with moving parts that are physically moved to change the displayed information), and FIGS. 6 and 7 show an example of a digital display. Other types of analogue or digital screen could be used. Alternatively or additionally, other indications could comprise one or more of a light such as an LED, a vibrating element, an electro-chromatic pigment, a noise-producing element such as a buzzer, a moveable arm or a protrusion configured to change position or extend once the set temperature is reached, for example. Where a physical movement of parts is needed, an electromagnet could be used, for example. In general, some form of indication is provided to the user by the feedback portion switching from a first state to a second state in response to the electronic circuit transitioning from the first mode to the second mode. The feedback portion may switch from a first state to a second state immediately in response to the electronic circuit transitioning from the first mode to the second mode or shortly afterwards. If the switch takes place shortly afterwards, it would typically be after a short delay (e.g. a delay of between 0.1 seconds and 5 minutes, or of between 2 and 10 seconds) between the electronic circuit transitioning from the first mode to the second mode and the feedback portion switching from a first state to a second state. Some of the indications above can be either reversible or irreversible, as described in more detail below. Instead of the electronic circuit comprising a feedback portion, a separate feedback portion could be provided, for example using a reversible or irreversible thermo-chromatic pigment.

A lock may be provided as part of the electronic circuit. If a lock is provided, the lock moves from a first state to a second state in response to the electronic circuit transitioning from the first mode to the second mode. For example, the lock is unlocked in the first state and locked in the second state, or unlocked in the second state and locked in the first state. As with the feedback portion, the lock may switch from a first state to a second state immediately in response to the electronic circuit transitioning from the first mode to the second mode or shortly afterwards. If the switch takes place shortly afterwards, it would typically be after a short delay (e.g. a delay of between 0.1 seconds and 5 minutes, or of between 2 and 10 seconds) between the electronic circuit transitioning from the first mode to the second mode and the lock switching from a first state to a second state.

A lock could be provided between a cap and an outer housing, between a needle cover and an outer housing or between a power pack and a housing, for example. As with the feedback portion, the lock could take various forms, such as a moveable clip, arm or protrusion hindering removal of the cap or the needle shield, and could be actuated by an electromagnet, for example. A lock could be provided in addition to a feedback portion, and could also be integrated with the feedback portion; for example, the movement of a feedback portion 30 such as that shown in FIGS. 4 and 5 could also unlock the device when the feedback portion 30 moves from the position in FIG. 4 to the position in FIG. 5. In such an example, the lock is locked in the first state and unlocked in the second state, to stop a user from injecting a drug until the drug is warm enough. Other examples are also possible; for example, the lock could be locked in the second state to stop a user from injecting a drug if the drug has been too warm or too cold at some point prior to the time of intended use. As with the provision of two or more feedback portions, two or more locks could also be provided. In one example with two locks, namely a first lock that is locked when the device is below a set temperature and unlocked when the device is above said set temperature, for example to stop a user using the device until the medicament is warm enough, and a second lock that is unlocked when the device is above (or alternatively below) a set temperature and is irreversibly locked if the device ever goes below (or alternatively above) said set temperature, for example to lock the device if the temperature goes too low and a drug freezes (or too high and the drug denatures). In this example, both locks will only be unlocked if the device has never been too cold (or alternatively too hot), in combination with the device currently being warm enough for the user to be allowed to inject the drug.

The change in the feedback portion can be reversible or irreversible. In a reversible feedback portion, the feedback portion can switch from a first state to a second state and then subsequently back to the first state again. One example would be a display that shows the message ‘READY’ as described in FIG. 7. Such a feedback portion is typically reversible because the display will turn off again if the temperature goes back below the set temperature, and can turn on again once the temperature goes back above the set temperature again. This can be useful where the primary intention is to indicate to a user whether the drug is warm enough to be injected. In contrast, an irreversible feedback portion would indicate whether the temperature has ever gone above the set temperature, either now or in the past. Such a feedback portion could optionally be provided using the example in FIGS. 4 and 5, for example, where a panel is moved from one position to another—the panel could be physically blocked from moving back into its first state (such as in FIG. 4) after it has moved to its second state (such as in FIG. 5). This could indicate whether a set temperature has ever been exceeded in the past, for example during transport of the device. A reversible feedback portion could be useful in terms of indicating whether a temperature warm enough for injection has been reached, for example, as it could allow the user to change their mind and put the device back in the fridge to be used later. For example, an irreversible feedback portion could be useful in terms of indicating whether the drug has been exposed to excessively high (or excessively low) temperatures that may cause the drug to lose efficacy, as an irreversible feedback portion would indicate that the device previously reached an unacceptable temperature, even after the device is back to an allowed temperature.

Similarly, in the event of a lock additionally or alternatively being provided, the lock can be reversible or irreversible. An example of an irreversible lock would be one that locks (or unlocks) when the temperature goes past the set temperature, and remains locked (or unlocked) even if the temperature subsequently goes back past the set temperature. An example of a reversible lock would be one that locks (or unlocks) when the temperature goes past the set temperature, and then unlocks (or locks) when the temperature subsequently goes back past the set temperature. Where both a feedback portion and a lock are provided, the feedback portion and the lock could both be irreversible or reversible, or only the feedback portion could be reversible with the lock being irreversible, or only the lock could be reversible with the feedback portion being irreversible.

The electronic circuit is configured so that it passively remains in a first mode when the temperature is on a first side of a set temperature and passively transitions to a second mode when the temperature transitions to a second side of the set temperature. For example, the first side of the set temperature could be below the set temperature, and the second side of the set temperature could be above the set temperature. In such as case, when the temperature is below a set temperature, the electronic circuit passively remains in the first mode, and when the temperature transitions to the second side of the set temperature, so in this case to above the set temperature, the electronic circuit transitions to the second mode. Alternatively, the first side of the set temperature could be above the set temperature, and the second side of the set temperature could be below the set temperature. In such a case, when the temperature is above a set temperature, the electronic circuit passively remains in a first mode, and when the temperature transitions to the second side of the set temperature, so in this case to below the set temperature, the electronic circuit transitions to a second mode. As discussed above in the examples for feedback portions and locks, the transition can result in irreversible changes or reversible changes, depending on the specific device design.

Alternatively or additionally to the provision of a lock and/or a feedback portion, the electronic circuit can include a recordal portion to record when the temperature transitions past (above or below) a set temperature. The recordal portion can be activated when the electronic circuit transitions from a first mode to a second mode, with power provided to run the recordal portion when the electronic circuit is in the second mode. In this way, a record can be kept of any periods when the device is above or below a certain temperature. For example, the recordal portion could record how many times a device has been above or below a certain temperature, and/or the recordal portion could record how long a device has been above or below a certain temperature. The recordal portion could comprise a microcontroller.

Once a device as described above has been assembled, including a medicament container containing a drug, it must normally be transported to a user for use. In an example with a drug that requires refrigeration, the medicament delivery device would typically be kept refrigerated for the entire shipping process, and would then be kept in a refrigerator by the user until use. Before the user uses the medicament delivery device, the medicament delivery device is removed from the refrigerator and is left to warm up. This allows the viscosity of the drug to decrease, which will typically make drug injection less painful. Once an appropriate set temperature been reached as described above, a device as described above can indicate to the user that the medicament delivery device is ready to be used by feedback from the feedback portion (and/or a lock unlocking), and the user can then inject the drug. In one example of this, the set temperature is 15 degrees Celsius, and the electronic circuit is configured to passively remain in a first mode below 15 degrees Celsius and to transition to a second mode once the temperature goes above 15 degrees Celsius. This could be suitable for a drug that requires refrigeration during transport and storage prior to use.

In an alternative embodiment of a device as described above, the electronic circuit is designed to indicate whether a set temperature has been exceeded at any point between assembly of the device and use of the device. In such cases, the feedback portion will indicate whether the temperature has ever increased above the set temperature as described above. The device can then be checked by a user, and the user would either be an end user checking before use, such as a patient or a caregiver, and/or a user checking at an intermediate point such as at some point during transport of the device between manufacture and use, where the user of the feedback given by the feedback portion could be someone other than an end user, for example a person involved in the supply chain or a pharmacist checking the device before providing it to a patient. In this way, the user can see whether the temperature has previously increased above the set temperature. In one example of this, the set temperature is 40 degrees Celsius, and the electronic circuit is configured to passively remain in a first mode below 40 degrees Celsius and to transition to a second mode once the temperature goes above 40 degrees Celsius.

In an alternative embodiment of a device as described above, the temperature sensor is on an outer portion of the device, such that the temperature sensor is able to measure the temperature of a user. In such a case, the electronic circuit would typically be configured to passively remain in a first mode when the temperature is below a set temperature and to transition to a second mode when the temperature transitions above the set temperature. As with the embodiments above, a feedback means and/or a lock can be provided. In one example, a feedback means could be provided that indicates whether the user is correctly holding a device, for example with the temperature sensor on the outer shell of a medicament delivery device and with a feedback means that is a light, for example where a green light is lit when the temperature of the sensor is above a specific temperature, for example 33 degrees Celsius. This would show the user when the device can be used. Alternatively or additionally to a feedback means, a lock could be provided that locks the medicament delivery device as discussed in more detail above, with the lock unlocking when the temperature sensor senses that the device is in contact with a user. In one example, the temperature sensor could be arranged on the medicament delivery member guard so as to sense the temperature of the user when the device is placed ready for injection, with the medicament delivery member guard touching the skin of the user. The lock could stop the medicament delivery member guard from being moved prior to the medicament delivery member guard being placed correctly for injection. In another example, the temperature sensor could be on an activation button of the device. This could result in the device being locked until the user presses the button, which could avoid activation of the device by an inanimate object, for example if the device is dropped on the floor. The opposite approach could also be taken, for example where a lock is unlocked in the first (passive) mode, and activates to lock the device if the user holds the device in the wrong way.

In general, the temperature sensor is on an outer portion of the device such that the temperature sensor is able to measure the temperature of a user, and as such could be placed anywhere on or in the device where the temperature sensor is able to measure the temperature of a user, for example by the user touching or holding the device. For example, this could be a housing, an outer shell, a button, a grip or a medicament delivery member guard of a medicament delivery device, or an outer housing, a button or a grip of a medicament delivery device add-on. When the temperature sensor is being used to measure the temperature of a user, the user is typically an end user, such as the user taking the medicament and/or the user administering the medicament using the device.

The description above mainly focusses on activating a feedback portion when the temperature has gone above a set temperature. This concept can also be used in the opposite direction, i.e. to indicate whether the temperature has dropped below a set temperature. This could be useful in cases where drug efficacy is affected by freezing of the drug, for example. In one example of this, the set temperature is 0 degrees Celsius, and the electronic circuit is configured to passively remain in a first mode above 0 degrees Celsius and to transition to a second mode once the temperature goes below 0 degrees Celsius. One way of achieving this would be to use a positive temperature coefficient thermistor (in which the resistance decreases when the temperature decreases) as the temperature sensor in an electronic circuit such as one of the electronic circuits described above.

As alluded to in the description above, more than one electronic circuit may be provided in combination in a medicament delivery device or medicament delivery device add-on according to the current disclosure. For example, a first electronic circuit may be provided to indicate to the user whether the device is ready to use, and a second electronic circuit may be provided to indicate to the user whether the device is still useable (i.e. whether the device has reached more than a certain temperature at any time after device manufacture, which would indicate whether the drug is still useable). The first and second electronic circuit could also be combined in a single electronic circuit. Additionally or alternatively, a third electronic circuit could be provided that keeps the device locked until a user has the proximal end of the medicament delivery member guard touching their skin, as determined by a temperature sensor on the proximal end of the medicament delivery member guard.

Various modifications to the embodiments described are possible and will occur to those skilled in the art without departing from the present disclosure which is defined by the following claims.

Some aspects of the present disclosure are described in the clauses below.

1. A device comprising an electronic circuit (20, 21), wherein the device comprises a medicament delivery device (10) and/or a medicament delivery device add-on,

the electronic circuit (20, 21) comprising a temperature sensor (T) configured to measure a temperature;

wherein the electronic circuit (20, 21) is configured to passively remain in a first mode when the temperature is on a first side of a set temperature and to transition to a second mode when the temperature transitions to a second side of the set temperature.

2. The device of clause 1, wherein the electronic circuit (20, 21) comprises a feedback portion (30, 34) configured to switch from a first state to a second state in response to the electronic circuit (20, 21) transitioning from the first mode to the second mode, so as to provide information related to the temperature to a user.

3. The device of clause 1 or 2, wherein the electronic circuit (20, 21) comprises a lock configured to switch from a first state to a second state in response to the electronic circuit (20, 21) transitioning from the first mode to the second mode.

4. The device of any of clauses 1 to 3, wherein the electronic circuit (20, 21) comprises a recordal portion to record when the electronic circuit transitions from the first mode to the second mode.

5. The device of any of clauses 1 to 4, wherein the electronic circuit (20, 21) is configured to passively remain in a first mode when the temperature is below a set temperature and to transition to a second mode when the temperature transitions to above the set temperature.

6. The device of any of clauses 1 to 4, wherein the electronic circuit (20, 21) is configured to passively remain in a first mode when the temperature is above a set temperature and to transition to a second mode when the temperature transitions to below the set temperature.

7. The device of any of clauses 2 to 6, wherein the feedback portion (30, 34) is configured to switch to the second state when the temperature is on the second side of the set temperature and is configured to return to the first state if the temperature subsequently returns to a temperature on the first side of the set temperature.

8. The device of any of clauses 2 to 6, wherein the feedback portion (30, 34) is configured to switch to the second state when the temperature is on the second side of the set temperature and is configured to remain in the second state if the temperature subsequently returns to a temperature on the first side of the set temperature.

9. The device of any of clauses 3 to 8, wherein the lock is configured to switch to the second state when the temperature is on the second side of the set temperature and is configured to return to the first state if the temperature subsequently returns to a temperature on the first side of the set temperature.

10. The device of any of clauses 3 to 8, wherein the lock is configured to switch to the second state when the temperature is on the second side of the set temperature and is configured to remain in the second state if the temperature subsequently returns to a temperature on the first side of the set temperature.

11. The device of any of clauses 1 to 10, wherein the medicament delivery device is an auto-injector or wherein the medicament delivery device add-on is an auto-injector add-on.

12. The device of any of clauses 3 to 11, wherein the lock is unlocked in the second state and locked in the first state.

13. The device of any of clauses 3 to 11, wherein the lock is unlocked in the first state and locked in the second state.

14. The device of any of clauses 1 to 13, wherein the temperature sensor (T) is configured to measure a temperature reflective of an ambient temperature, of a temperature of the device, of a medicament within the device, or of a user.

15. The device of any of clauses 1 to 14, wherein the temperature sensor is on an outer portion of the device.

16. The device of clause 15, wherein the temperature sensor is configured to measure the temperature of a user.

17. The device of clause 15 or 16, wherein the device comprises a medicament delivery device, and wherein the outer portion of the device is part of a housing, an outer shell, a button, a grip or a medicament delivery member guard of the medicament delivery device.

18. The device of clause 15 or 16, wherein the device comprises a medicament delivery device add-on, and wherein the outer portion of the device is part of an outer housing, a button or a grip of the medicament delivery device add-on.

19. The device of clause 17, wherein the outer portion of the device is a medicament delivery member guard of the medicament delivery device, wherein the medicament delivery member guard extends from a proximal end to a distal end, wherein the proximal end is the end closest to the injection site when in use, and wherein the temperature sensor is at the proximal end of the medicament delivery member guard.

20. The device of any of clauses 15 to 19, wherein the set temperature is a temperature between 20 and 37 degrees Celsius, or between 25 and 35 degrees Celsius, or between 30 and 33 degrees Celsius.

21. The device of any of clauses 1 to 14, wherein the set temperature is a temperature between −50 and 100 degrees Celsius.

22. The device of clause 21, wherein the set temperature is a temperature between −10 and 10 degrees Celsius, between 5 and 25 degrees Celsius, or between 25 and 50 degrees Celsius.

23. The device of any of clauses 1 to 22, wherein the temperature sensor (T) is a thermistor.

24. The device of any of clauses 1 to 23, wherein the electronic circuit (20, 21) comprises a voltage comparator.

25. The device of clause 24, wherein the voltage comparator comprises one or more of a transistor (TR) and an operational amplifier (A).

26. The device of any of clauses 1 to 25, wherein the feedback portion provides one or more of tactile, audible and visual feedback.

27. The device of any of clauses 1 to 26, wherein the feedback portion comprises one or more of a display, a buzzer, a light and a switch.

28. The device of clause 1, wherein the set temperature is a temperature above which a drug may be injected.

29. The device of clause 1, wherein the set temperature is a temperature above which or a temperature below which a drug is no longer useable.

30. An electronic unit of a medicament delivery device or a medicament delivery device add-on, the electronic unit comprising a temperature sensor configured to measure a temperature, the electronic unit being configured to passively remain in a first mode when the temperature is on a first side of a set temperature and to transition to a second mode when the temperature transitions to a second side of the set temperature.

31. The electronic unit of clause 30, wherein the electronic unit comprises a feedback portion configured to switch from a first state to a second state in response to the electronic unit transitioning from the first mode to the second mode, so as to provide information related to the temperature to a user; and/or a lock configured to switch from a first state to a second state in response to the electronic unit transitioning from the first mode to the second mode, and/or a recordal portion to record when the electronic circuit transitions from the first mode to the second mode.

1-30. (canceled)

31. A device comprising an electronic circuit, wherein the device comprises a medicament delivery device and/or a medicament delivery device add-on,
the electronic circuit comprising a temperature sensor configured to measure a temperature;
wherein the electronic circuit is configured to passively remain in a first mode when the temperature is on a first side of a set temperature and to transition to a second mode when the temperature transitions to a second side of the set temperature.

32. The device of claim 31, wherein the electronic circuit comprises a feedback portion configured to switch from a first state to a second state in response to the electronic circuit transitioning from the first mode to the second mode, so as to provide information related to the temperature to a user.

33. The device of claim 31, wherein the electronic circuit comprises a lock configured to switch from a first state to a second state in response to the electronic circuit transitioning from the first mode to the second mode.

34. The device of any of claim 31, wherein the electronic circuit comprises a recordal portion to record when the electronic circuit transitions from the first mode to the second mode.

35. The device of any of claim 31, wherein the electronic circuit is configured to passively remain in a first mode when the temperature is below a set temperature and to transition to a second mode when the temperature transitions to above the set temperature.

36. The device of any of claim 31, wherein the electronic circuit is configured to passively remain in a first mode when the temperature is above a set temperature and to transition to a second mode when the temperature transitions to below the set temperature.

37. The device of claim 32, wherein the feedback portion is configured to switch to the second state when the temperature is on the second side of the set temperature and is configured to return to the first state if the temperature subsequently returns to a temperature on the first side of the set temperature.

38. The device of claim 32, wherein the feedback portion is configured to switch to the second state when the temperature is on the second side of the set temperature and is configured to remain in the second state if the temperature subsequently returns to a temperature on the first side of the set temperature.

39. The device of claim 33, wherein the lock is configured to switch to the second state when the temperature is on the second side of the set temperature and is configured to return to the first state if the temperature subsequently returns to a temperature on the first side of the set temperature.

40. The device of claim 33, wherein the lock is configured to switch to the second state when the temperature is on the second side of the set temperature and is configured to remain in the second state if the temperature subsequently returns to a temperature on the first side of the set temperature.

41. The device of claim 31, wherein the medicament delivery device is an auto-injector or wherein the medicament delivery device add-on is an auto-injector add-on.

42. The device of claim 33, wherein the lock is unlocked in the second state and locked in the first state.

43. The device of claim 33, wherein the lock is unlocked in the first state and locked in the second state.

44. The device of claim 31, wherein the temperature sensor is configured to measure a temperature reflective of an ambient temperature, of a temperature of the device, of a medicament within the device, or of a user.

45. The device of claim 31, wherein the temperature sensor is on an outer portion of the device.

46. The device of claim 45, wherein the temperature sensor is configured to measure the temperature of a user.

47. The device of claim 45, wherein the device comprises a medicament delivery device, and wherein the outer portion of the device is part of a housing, an outer shell, a button, a grip or a medicament delivery member guard of the medicament delivery device.

48. The device of claim 45, wherein the device comprises a medicament delivery device add-on, and wherein the outer portion of the device is part of an outer housing, a button or a grip of the medicament delivery device add-on.

49. The device of claim 47, wherein the outer portion of the device is a medicament delivery member guard of the medicament delivery device, wherein the medicament delivery member guard extends from a proximal end to a distal end, wherein the proximal end is the end closest to the injection site when in use, and wherein the temperature sensor is at the proximal end of the medicament delivery member guard.

50. The device of claim 31, wherein the electronic circuit is configured to passively remain in a first mode without periodically measuring the temperature.
